(19) United States
(12) Patent Application Publication
(10) Pub. No.: US 2023/0238083 A1
(43) Pub. Date: Jul. 27, 2023

(54) SINGLE-STEP RETROSYNTHESIS METHOD AND SYSTEM BASED ON MULTI-SEMANTIC NETWORK

(71) Applicant: WUHAN UNIVERSITY, Wuhan City, (CN)

(72) Inventors: Juan LIU, Wuhan City (CN); Qiang ZHANG, Wuhan City (CN); Feng YANG, Wuhan City (CN); Zhihui YANG, Wuhan City (CN)

(21) Appl. No.: 18/095,586

(22) Filed: Jan. 11, 2023

(30) Foreign Application Priority Data
Jan. 24, 2022 (CN) 202210080029.X

(51) Int. Cl. G16C 20/10; G16C 20/70

(52) U.S. Cl. CPC G16C 20/10; G16C 20/70

(57) ABSTRACT

A single-step retrosynthesis method and system based on a multi-semantic network are provided. The method includes the following steps: inputting an ECFP4 feature and a SMILES word one-hot feature of a target product molecule during the single-step retrosynthesis prediction, and outputting the first k reactions which may occur on the target product molecule in a reaction template through the multi-semantic network. The SMILES string of a reactant corresponding to the target product molecule is calculated by applying the output reaction template into SMILES string of the target product molecule. The present disclosure is the first method for performing single-step retrosynthesis prediction by using a multi-semantic fusion network in the field of single-step retrosynthesis. It is a template-based single-step retrosynthesis method, and the prediction result has relatively high interpretability. The network learns fused semantic features, ECFP4 semantic features and SMILES word one-hot semantic features.

### CROSS REFERENCE TO RELATED APPLICATION

This patent application claims the benefit and priority of Chinese Patent Application No. 202210080029.X filed with the China National Intellectual Property Administration on Jan. 24, 2022, the disclosure of which is incorporated by reference herein in its entirety as part of the present application.

### TECHNICAL FIELD

The present disclosure relates to the analysis field of single-step retrosynthesis in retrosynthesis, and in particular to a single-step retrosynthesis method and system based on a multi-semantic network, which belongs to the application of neural network models of machine learning in the field of single-step retrosynthesis.

### BACKGROUND

The core idea of retrosynthesis is to recursively decompose target molecules into simple and available precursor molecules until they are commercially available or appear in chassis biological strains. These molecules can be obtained by chemical or enzymatic reactions. Therefore, retrosynthesis can be divided into chemical retrosynthesis and bioretrosynthesis according to different types of molecule reactions. Imperfect professional background knowledge and huge search space enable the retrosynthesis to be a huge challenge. In this case, computer-aided retrosynthesis path design methods are gradually being deployed and have great development potential, which also brings the possibility to synthesize new high-value molecules.

Up to now, methods of chemical retrosynthesis are mature, but these methods have failed to consider a principle of green chemistry: the use of raw materials, catalysts, solvents and reagents, products, by-products, etc. are easily harmful to the ecological environment and community safety. Based on this, finding green and environmentally friendly retrosynthesis paths has been widely concerned by experts and scholars. At the beginning of the 21st century, the concept of bioretrosynthesis was first put forward. Bioretrosynthesis recursively decomposes the target molecules into “precursor” molecules and limits the reactions to the enzymatic reactions until the final starting molecules are all available precursors in the chassis biological strains. Compared with the traditional chemical retrosynthesis method, bioretrosynthesis is safer and more environmentally friendly and saves cost.

The existing retrosynthesis methods all need to solve a problem: the single-step retrosynthesis problem, which predicts possible reactants based on intermediates. The whole retrosynthesis path design is optimized by optimizing the single-step retrosynthesis. The description of molecules in the retrosynthesis method mainly comprises ECFP4 (Extended Connectivity Fingerprints) vector and SMILES (Simplified Molecular Input Line Entry Specification). First, the ECFP4 vector characterizes a count of all functional groups in a reaction process, and can represent complex relationship between atoms in the functional groups, but cannot represent the sequential relationship between atoms. The existing retrosynthesis methods use ECFP4 vector as input which is mainly composed of template-based methods. The retrosynthesis is problem is transformed into a classification problem from molecule fingerprints to reaction templates. These methods leverage the similarity of ECFP4 vectors or use simple neural networks, which leads to insufficient learning of effective information of the ECFP4 vectors. The molecular SMILES string represents a hypothetical sequential relationship between atoms, but cannot represent the complex relationship between atoms. Another retrosynthesis methods are template-free methods that use molecule SMILES strings as input. These methods convert the single-step retrosynthesis problem into a sequence translation problem, but these methods have poor interpretability. Moreover, the existing template-free methods cannot match enzyme information.

Therefore, the prior art has a problem of poor prediction performance.

### SUMMARY

Aiming at defects of the prior art mentioned in the background, the present disclosure provides a method and system of a multi-semantic network for single-step retrosynthesis. The method regards the single-step inverse retrosynthesis problem as a multi-classification problem and then makes the retrosynthesis prediction.

The technical solution adopted by the present disclosure is as follows.

The first aspect provides a single-step retrosynthesis method based on a multi-semantic network, which includes:

S1: acquiring a public data set, and preprocessing the public data set to obtain a preprocessed data set D, wherein each piece of data in the data set D corresponds to one specific reaction, and each piece of data comprises a reaction, a reactant molecule and a product molecule;

S2: extracting reaction templates from all reactions in the data set D by using an RDChiral tool, and removing repeated reaction templates, to obtain a final reaction template set T, wherein each reaction template corresponds to one or more reactions;

S3: obtaining an ECFP4 feature set E of product molecules represented by an ECFP4 vector and a SMILES word one-hot feature set S of the product molecules represented by a SMILES word one-hot matrix, respectively, according to the product molecules in the data set D;

S4: constructing a sample setsG={(eᵢ, sᵢ), tᵢ}ᵢ₌₁^{N}, where eᵢ∈E and sᵢ∈S represent the ECFP4 feature and the SMILES word one-hot feature of a product molecule in a i-th data of the data set D, respectively, tᵢ∈T represents a reaction template in the i-th data of the data set D, and N represents a number of the sample set;

S5, constructing the multi-semantic network, wherein the multi-semantic network comprises an input layer, a convolution layer, a normalization layer, an activation layer, a pooling layer, a dropout layer, a fully connected layer and an output layer, wherein the convolution layer is configured to convolve input data, the normalization layer is configured to normalize a convolved feature, the activation layer is configured to activate a normalized feature, and the pooling layer performs pooling operation on the ECFP4 feature and the SMILES word one-hot feature, respectively, so as to obtain an ECFP4 semantic feature and a SMILES word one-hot semantic feature; fusing the ECFP4 semantic feature with the SMILES word one-hot semantic feature to obtain a fused semantic feature; and passing the fused semantic features through the dropout layer, the fully connected layer and Softmax, to obtain a final output result by the output layer;

S6: training the multi-semantic network in S5 by using the sample sets in S4 to obtain a trained single-step retrosynthesis prediction model;

S7: for a target product molecule to be predicted, predicting a reaction template capable of generating the target product molecule by using the trained single-step retrosynthesis prediction model trained in S6, and calculating a SMILES string of the reactant molecule corresponding to the target product molecule by using the RDChiral tool in combination with the SMILES string of the target product molecule, thereby realizing single-step retrosynthesis prediction.

In an embodiment, the S2 comprises:

extracting reaction templates from all the data in the data set D by using an RDChiral tool to obtain a final reaction template set T, and removing repeated reaction templates.

In an embodiment, the S3 comprises:

according to the product molecule in the data set D, generating the ECFP4 vector of the product molecule in all the data in the data set D by using the RDKit tool to obtain the ECFP4 feature set E of the product molecule represented by the ECFP4 vectors; generating the SMILES word one-hot matrix of the product molecule in all the data in the data set D by using a Sklearn tool to obtain the SMILES word one-hot feature set S of the product molecules represented by the SMILES word one-hot matrix.

In an embodiment, the generating the SMILES word one-hot matrix of the product molecule in all the data in the data set D by using a Sklearn tool to obtain the SMILES word one-hot feature set S of the product molecules represented by the SMILES word one-hot matrix comprises:

S3.1: performing one-hot-encoding on each character of an alphabet constructing the SMILES string to generate a word vector with dimension w₂; using word vectors of a first l₂ characters in each product molecule SMILES string to form a SMILES word one-hot matrix s₂∈{0,1}^{l}^{₂}^{×w}^{₂}, wherein if the SMILES string of the product molecule has less than l₂ characters, the product molecule SMILES is padded with 0 vector;

S3.2: deeming every successive n rows in the matrix s₂∈{0,1}^{l}^{₂}^{×w}^{₂} as a group, the n rows corresponding to the word vectors of n characters, concatenating the word vectors in the same group in sequence to obtain a composition of word vector with a length of w₁, w₁=n*w₂, w₂, wherein a total of l₁ composition of word vectors being obtained,

$${l}_{1} = \frac{{l}_{2}}{n} ,$$

and constituting the SMILES word one-hot feature, of the product molecules∈{0,1}^{l}^{₁}^{×w}^{₁}, where w₂, l₂ and n are positive integers, and n<l₂.

In an embodiment, the multi-semantic network in S5 has one input layer, k₁+k₂ convolution layers, k₁+k₂ normalization layers, k₁+k₂ activation layers, k₁+k₂ pooling layers, two dropout layers, three fully connected layers and three output layers, where k₁ and k₂ are positive integers,

the processing step in S5 comprises:

S5.1: inputting the ECFP4 feature represented by the ECFP4 vector at the input node 1, and inputting the SMILES word one-hot feature represented by the SMILES word one-hot matrix at input node 2, wherein the input layer including the input node 1 and the input node 2;

S5.2: convolving the ECFP4 feature input at the input node 1 by using k₁ convolution kernels with a same size to obtain a convolved ECFP4 feature, wherein a number of output channels of the k₁ convolution kernels is c₁;

S5.3: convolving the SMILES word one-hot feature input at the input node 2 by using k₂ convolution kernels with different sizes to obtain a convolved SMILES word one-hot feature, wherein a number of output channels of the k₂ convolution kernels is c₂;

S5.4: normalizing the convolved features in S5.2 and convolved features in S5.3 by the normalization layer, respectively, to obtain a normalized ECFP4 feature and a normalized SMILES word one-hot feature;

S5.5: performing ReLU activation operation on the normalized ECFP4 feature and the normalized SMILES word one-hot feature by the activation layer, respectively, to obtain an activated ECFP4 feature and an activated SMILES word one-hot feature;

S5.6: performing Max-Pooling operation on the activated ECFP4 feature and the activated SMILES word one-hot feature by the pooling layer, respectively, to obtain a pooled ECFP4 feature and a pooled SMILES word one-hot feature;

S5.7: concatenating the pooled ECFP4 feature to obtain concatenated ECFP4 semantic feature, concatenating the pooled SMILES word one-hot feature to obtain a concatenated SMILES word one-hot semantic feature, and concatenating the ECFP4 semantic feature and the SMILES word one-hot semantic feature to obtain fused semantic features;

S5.8: feeding the fused semantic features to one fully connected layer, and passing the fused semantic features through Softmax, to output probability of each node which falls between [0,1] and is denoted as p₁∈R^{d}, p₁ indicating a predicted occurrence probability of each type of reaction templates; passing the ECFP4 semantic feature and the SMILES word one-hot semantic feature through the dropout layer, the fully connected layer and Softmax, respectively, to output probabilities of each node which fall between [0,1] are denoted as p₂∈R^{d} and p₃∈R^{d}, respectively, p₂ and p₃ indicating occurrence probabilities of each type of reaction templates predicted according to the ECFP4 semantic feature and SMILES word one-hot semantic feature, d is the number of the reaction templates in a reaction template set T;

S5.9: obtaining a final predicted result by the output layer according to a result in S5.8.

In an embodiment, in the training process of step S6, according to three classification results of the model, three cross entropy losses of the model are denoted as loss₁, loss₂ and loss₃, respectively, and a final loss of the single-step retrosynthesis prediction model is:

loss=α₁loss₁+α₂loss₂+α₃loss₃

where loss₁, loss₂ and loss₃ represent a predicted loss of the fused semantic feature, a predicted loss of the ECFP4 semantic feature and a predicted loss of the SMILES word one-hot semantic feature, respectively, and αⱼ (j=1,2,3) represents a weight of the three losses loss₁, loss₂ and loss₃ in the global loss of the network, respectively, where Σαⱼ=1 and αⱼ∈(0,1).

Based on the same inventive concept, a second aspect of the present disclosure provides a single-step retrosynthesis system based on a multi-semantic network, comprising:

a data set preprocessing module configured to acquire a public data set and preprocess the public data set to obtain a preprocessed data set D, wherein each piece of data in the data set D corresponds to one specific reaction, and each piece of data comprises a reaction, a reactant molecule and a product molecule;

a reaction template set constructing module configured to construct a reaction template set T;

a feature constructing module configured to obtain an ECFP4 feature set E of the product molecules represented by an ECFP4 vector and a SMILES word one-hot feature set S of the product molecules represented by a SMILES word one-hot matrix, respectively, according to the target product molecules in the data set D;

a sample set constructing module configured to construct a sample sets G={(eᵢ, sᵢ), tᵢ}ᵢ₌₁^{N}, where eᵢ∈E and sᵢ∈S represent the ECFP4 feature and the SMILES word one-hot feature of a product molecule in a i-th data of the data set D, respectively, tᵢ∈T represents a reaction template in the i-th data of the data set D, and N represents a number of sample sets;

a multi-semantic network constructing module configured to construct the multi-semantic network, wherein the multi-semantic network comprises an input layer, a convolution layer, a normalization layer, an activation layer, a pooling layer, a dropout layer, a fully connected layer and an output layer, the convolution layer is configured to convolve input data, the normalization layer is configured to normalize a convolved feature, the activation layer is configured to activate a normalized feature, and the pooling layer performs pooling operation on the ECFP4 feature and the SMILES word one-hot feature, respectively, so as to obtain the ECFP4 semantic feature and the SMILES word one-hot semantic feature; fuse the ECFP4 semantic feature with the SMILES word one-hot semantic feature to obtain a fused semantic features; and pass the fused semantic features through the dropout layer, the fully connected layer and Softmax, to obtain a final output result by the output layer;

a multi-semantic network training module configured to train the multi-semantic network in the multi-semantic network constructing module by using the sample set of the sample set constructing module to obtain a trained single-step retrosynthesis prediction model;

a single-step retrosynthesis predicting module configured to, for a target product molecule to be predicted, predict a reaction template capable of generating the target product molecule by using the trained single-step retrosynthesis prediction model in the multi-semantic network training module, and calculate the SMILES string of the reactant corresponding to the target product molecule in combination with the SMILES string of the target product molecule, thereby realizing single-step retrosynthesis prediction.

The above one or more technical solutions in the embodiments of the present disclosure have at least one or more of technical effects as follows.

The single-step retrosynthesis method based on the multi-semantic network according to the present disclosure constructs a multi-semantic network, a multi-semantic network comprises an input layer, a convolution layer, a normalization layer, an activation layer, a pooling layer, a dropout layer, a fully connected layer and an output layer. The ECFP4 feature and the SMILES word one-hot feature of the target product molecule are used as inputs. The deep semantic feature of the ECFP4 feature and the SMILES word one-hot feature of the target product molecule can be extracted by a semantic extraction method. Compared with the simple neural network method, the single-step retrosynthesis method can fully learn the effective information of the ECFP4 feature and the SMILES one-hot feature, and learn complementary information by fusing semantics, thus improving prediction results. In addition, the present disclosure adopts a template-based single-step retrosynthesis method, and the prediction result has relatively high interpretability. In addition, the single-step retrosynthesis prediction model of the present disclosure can be used as both single-step chemical retrosynthesis prediction and single-step bioretrosynthesis prediction, and matching operation of enzyme information is not required when the single-step bioretrosynthesis prediction is carried out.

### BRIEF DESCRIPTION OF THE DRAWINGS

In order to explain the embodiments of the present disclosure or the technical solutions in the prior art more clearly, drawings used in the description of the embodiments or the prior art will be briefly described below. Apparently, the drawings in the following description are some embodiments of the present disclosure. For those skilled in the art, other drawings can be obtained according to these drawings without any creative labor.

FIG. 1 is a flow chart of single-step retrosynthesis prediction of a multi-semantic network according to an embodiment of the present disclosure.

FIG. 2 is a diagram of a SMILES word one-hot feature according to an embodiment of the present disclosure.

FIG. 3 is a schematic diagram of a multi-semantic network according to an embodiment of the present disclosure.

FIG. 4 is a schematic diagram of a single-step retrosynthesis system module of a multi-semantic network according to an embodiment of the present disclosure.

### DETAILED DESCRIPTION OF THE EMBODIMENTS

The present disclosure discloses a single-step retrosynthesis method and system based on a multi-semantic network, the method includes the following steps: inputting an ECFP4 feature and a SMILES word one-hot feature of a target product molecule during the single-step retrosynthesis prediction, and outputting a first k reactions which may occur on the target product molecule in a reaction template form after passing through the multi-semantic network. The SMILES string of the reactant corresponding to the target product molecule is finally calculated according to the output reaction template and in combination with the SMILES string of the target product molecule, thereby realizing single-step retrosynthesis prediction. The present disclosure further provides a single-step retrosynthesis system based on a multi-semantic network, which performs single-step retrosynthesis prediction by preprocessing a data set, constructing a reaction template set, constructing a feature, constructing a sample set, constructing a multi-semantic network, and training a multi-semantic network.

The method and system of embodiments of the present disclosure have the following advantages or beneficial technical effects.

The single-step retrosynthesis prediction method is a first method for performing single-step retrosynthesis prediction by using a multi-semantic fusion network in the field of single-step retrosynthesis, and is a template-based single-step retrosynthesis method. The prediction result has relatively high interpretability. The present disclosure designs a new loss function, which can improve the training precision of the model. The present disclosure designs a semantic extraction method, which can extract a deep semantic feature of the ECFP4 feature and the SMILES word one-hot feature of the target product molecule. The present disclosure designs a construction method of a SMILES word one-hot feature, which can contain more potential information. The single-step retrosynthesis prediction model of the present disclosure can be used as both single-step chemical retrosynthesis prediction and single-step bioretrosynthesis prediction, and matching operation of enzyme information is not required when the single-step bioretrosynthesis prediction is carried out.

In order to make the purposes, technical solutions and advantages of the embodiments of the present disclosure clearer, the technical solutions in the embodiment of the present disclosure will be clearly and completely described below with reference to the drawings in the embodiments of the present disclosure. Apparently, described embodiments are a part of the embodiments of the present disclosure, rather than all of the embodiments. Based on the embodiment of the present disclosure, all other embodiments obtained by those skilled in the art without any creative labor fall within protection scope of the present disclosure.

### Embodiment 1

The embodiment of the present disclosure provides a single-step retrosynthesis method based on a multi-semantic network, which includes the following steps S1-S7.

In step S1, a public data set is acquired and preprocessed to obtain a preprocessed data set D. Each piece of data in the data set D corresponds to one specific reaction, and each piece of data includes a reaction, a reactant and a target product molecule.

In step S2, reaction templates are extracted from all the data in the data set D, and repeated reaction templates are removed by using an RDChiral tool to obtain a final reaction template set T. Each reaction template contains one or more reactions.

In step S3, according to the product molecule in the data set D, an ECFP4 feature set E of a product molecule represented by an ECFP4 vector and a SMILES word one-hot feature set S of a product molecule represented by a SMILES word one-hot matrix are obtained, respectively.

In step S4, a sample set G={(eᵢ, sᵢ), tᵢ}ᵢ₌₁^{N} is constructed, where eᵢ∈E and sᵢ∈S represent the ECFP4 feature and the SMILES word one-hot feature of a product molecule in a i-th data of the data set D, respectively, tᵢ∈T represents a reaction template in the i-th data of the data set D, and N represents a number of sample sets.

In step S5, a multi-semantic network is constructed, the multi-semantic network includes an input layer, a convolution layer, a normalization layer, an activation layer, a pooling layer, a dropout layer, a fully connected layer and an output layer. The convolution layer is configured to perform convolution on the input data, the normalization layer is configured to perform normalization on the convolved feature, the activation layer is configured to activate the normalized feature, and the pooling layer performs pooling operation on the ECFP4 feature and the SMILES word one-hot feature, respectively, so as to obtain an ECFP4 semantic feature and a SMILES word one-hot semantic feature. The ECFP4 semantic feature is fused with the SMILES word one-hot semantic feature to obtain a fused semantic feature; and then, the fused semantic feature is passed through the dropout layer, the fully connected layer and Softmax, to obtain a final output result outputted by the output layer.

In step S6, the multi-semantic network in step S5 is trained by using the sample set in step S4 to obtain a trained single-step retrosynthesis prediction model.

In step S7, for a target product molecule to be predicted, the reaction template capable of generating the target product molecule is predicted using the single-step retrosynthesis prediction model trained in step S6, and then the SMILES string of the reactant molecule corresponding to the target product molecule is calculated by using the RDChiral tool in combination with the SMILES string of the target product molecule, thereby realizing single-step retrosynthesis prediction.

In the specific implementation process, each piece of data in the data set D specifically includes: (1) reaction represented by the SMILES string; (2) all reactant molecules participating in the reaction (represented by the SMILES string, in which if there is more than one reactant molecule, the SMILES strings of a plurality of reactants are separated by separators); (3) one product molecule generated by the reaction (represented by the SMILES string); (4) number of catalytic enzyme (only for metabolic reaction, which is not necessary). If there are a plurality of product molecules for a certain reaction in the original public data set, there are a plurality of related data in D, and each data corresponds to one product of the reaction.

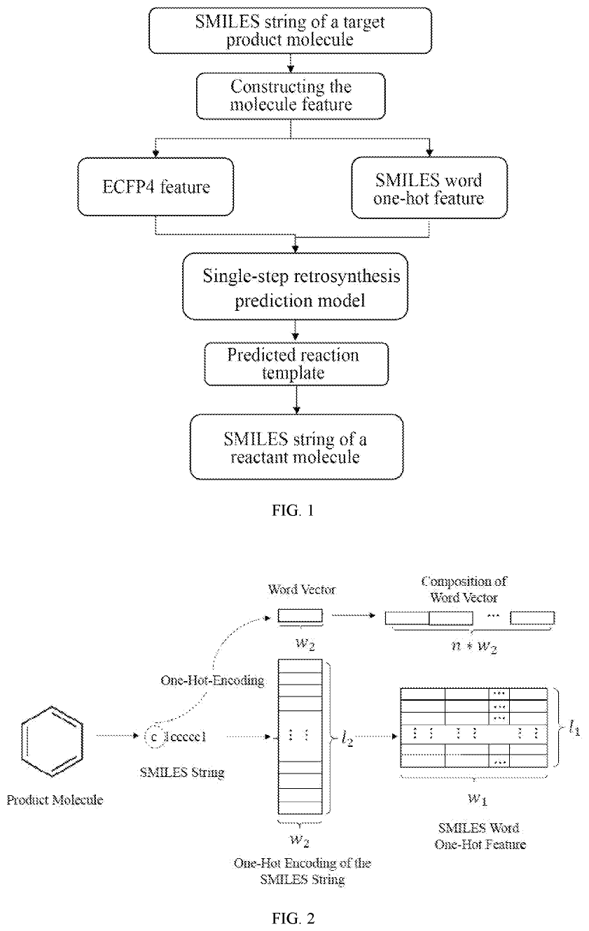

Referring to FIG. 1, a flow chart of single-step retrosynthesis prediction of a multi-semantic network according to an embodiment of the present disclosure is shown.

In step S7, during the synthesis prediction of the target product molecule to be predicted, an ECFP4 feature and a SMILES word one-hot feature of a target product molecule to be predicted are input, and the first k reactions which may occur on the target product molecule are output in a reaction template form through a single-step retrosynthesis prediction model. The SMILES string of the reactant corresponding to the target product molecule can be finally calculated according to the reaction template in combination with the SMILES string of the target product molecule, thereby realizing single-step retrosynthesis prediction.

In an embodiment, step S2 includes:

extracting reaction templates from all the data in the data set D and removing repeated reaction templates by using an RDChiral tool to obtain a final reaction template set T.

In the specific implementation process, the template_extractor function (template extraction function) in RDChiral is used to extract a reaction template in a SMARTS format.

In an embodiment, step S3 includes:

according to the product molecule in the data set D, generating an ECFP4 vector of the product molecule in all the data in the data set D by using the RDKit tool to obtain an ECFP4 feature set E of the product molecule represented by the ECFP4 vector; generating a SMILES word one-hot matrix of the product molecule in all the data in the data set D using a Sklearn tool to obtain the SMILES word one-hot feature set S of the product molecule represented by the SMILES word one-hot matrix.

In an embodiment, generating a SMILES word one-hot matrix of the product molecule in all the data in the data set D using a Sklearn tool to obtain the SMILES word one-hot feature set S of the product molecule represented by the SMILES word one-hot matrix includes the following steps S3.1-S3.2.

In step S3.1, one-hot-encoding is performed on each character of alphabet for constructing the SMILES string, to generate a word vector with a dimension of w₂; the word vector of the first l₂ characters in each product molecule SMILES string is taken to form a SMILES word one-hot matrix s₂∈{0,1}^{l}^{₂}^{×w}^{₂}, and if the product molecule SMILES string has less than l₂ characters, it is padded with 0 vector.

In step S3.2, every successive n rows in the matrix s₂∈{0,1}^{l}^{₂}^{×w}^{₂} is taken as a group, in which the n rows correspond to the word vectors of n characters, the word vectors in the same group are concatenated in sequence to obtain a composition of word vector with a length of w₁, w₁=n*w₂, a total of l₁ composition of word vectors are obtained,

$${l}_{1} = \frac{{l}_{2}}{n} ,$$

thereby constituting the SMILES word one-hot feature of the product molecule s∈{0,1}^{l}^{₁}^{×w}^{₁}, where w₂, l₂ and n are positive integers, and n<l₂.

Specifically, the SMILES word one-hot feature of the product molecule is a 0-1 matrix, and each row of the matrix represents the composition of word vector representation of the consecutive n characters in the SMILES string of the product molecule. The SMILES word one-hot feature of the product molecule is generated by the following method. First, it is assumed that the alphabet of all molecule SMILES strings in the data set D contains w₂ letters in total, and one-hot-encoding is performed on each character in all SMILES strings, so as to generate a word vector with length w₂. Thereafter, all the first l₂ characters in the product molecule SMILES string are represented by word vectors, and the product molecule SMILES string with less than l₂ characters are padded with 0 vector to obtain the matrix s₂∈{0,1}^{l}^{₂}^{×w}^{₂}. Finally, starting from the first row, every consecutive n rows (word vectors of n characters) in the matrix s₂∈{0,1}^{l}^{₂}^{×w}^{₂} are concatenated into a composition of word vector with length w₁ (w₁=n*w₂) in sequence to obtain a total of

$${l}_{1} ( {l}_{1} = \frac{{l}_{2}}{n} )$$

composition of word vectors, and the matrix formed by the composition of word vectors is the SMILES one-hot matrix feature of the product molecule s∈{0,1}^{l}^{₁}^{×w}^{₁}, where w₂, l₂ and n are positive integers, and n<l₂.

In the specific implementation process, the length of the ECFP4 feature of the product molecule is 4096. The dimension of the SMILES word one-hot feature of the product molecule is s∈{0,1}^{75×120}, which is a feature of vectorization representation the product molecule SMILES string. The generating step includes the following steps. First, one-hot-encoding is performed on the alphabet consisted of characters in all the molecule SMILES strings, and the 40-dimension word vector is generated. Then, the first 225 characters in the product molecule SMILES string are all represented by word vectors, and the product molecule SMILES string with less than 225 characters are padded with 0 vector to obtain s₂∈{0,1}^{225×40}. Finally, the word vectors of every three consecutive characters in s₂∈{0,1}^{225×40} are concatenated into a composition of word vector to finally obtain the product word one-hot matrix s∈{0,1}^{75×120.}

In an embodiment, the multi-semantic network in step S5 has one input layer, k₁+k₂ convolution layers, k₁+k₂ normalization layers, k₁+k₂ activation layers, k₁+k₂ pooling layers, two dropout layers, three fully connected layers and three output layers, where k₁ and k₂ are positive integers,

The processing step includes the following steps S5.1-S5.9.

In step S5.1, the input layer includes two nodes, the ECFP4 feature represented by the ECFP4 vector is input at the input node 1, and the SMILES word one-hot feature represented by the SMILES word one-hot matrix is input at the input node 2.

In step S5.2, the ECFP4 feature input at the node 1 is convolved by using k₁ convolution kernels with the same size, in which a number of output channels of k₁ convolution kernels is c₁, so as to obtain the convolved ECFP4 feature.

In step S5.3, the SMILES word one-hot feature input at the node 2 is convolved by using k₂ convolution kernels with different sizes, in which a number of output channels of k₂ convolution kernels is c₂, so as to obtain the convolved SMILES word one-hot feature.

In step S5.4, the feature after convolution in S5.2 and the feature after convolution in S5.3 are normalized by the normalization layer, respectively, to obtain the normalized ECFP4 feature and the SMILES word one-hot feature.

In step S5.5, ReLU activation operation is performed on the normalized ECFP4 feature and the SMILES word one-hot feature by the activation layer, respectively, to obtain the activated ECFP4 feature and the activated SMILES word one-hot feature.

In step S5.6, max-pooling operation is performed on the activated ECFP4 feature and the activated SMILES word one-hot feature by the pooling layer, respectively, to obtain the ECFP4 feature and the SMILES word one-hot feature after pooling operation.

In step S5.7, the ECFP4 feature after max-pooling is concatenated to obtain the concatenated ECFP4 semantic feature, the SMILES word one-hot feature after max-pooling is concatenated to obtain the concatenated SMILES word one-hot semantic feature, and the ECFP4 semantic feature and the SMILES word one-hot semantic feature are concatenated to obtain the fused semantic features.

In step S5.8, the fused semantic features are fed to a fully connected layer, and then passed through Softmax to output the probability of each node which falls between [0,1] and is denoted as p₁∈R^{d}, such probability indicates the predicted occurrence probability of each type of reaction templates. Furthermore, the ECFP4 semantic feature and the SMILES word one-hot semantic feature are passed through the dropout layer, the fully connected layer and Softmax, respectively, so as to output the probabilities of each node which fall between [0, 1] and are denoted as p₂∈R^{d} and p₃∈R^{d}, respectively. p₂ and p₃ indicate the occurrence probabilities of each type of reaction templates predicted according to the ECFP4 semantic feature and SMILES word one-hot semantic feature respectively. d is a number of the reaction templates in a reaction template set T.

In step S5.9, the final predicted result is obtained by the output layer according to the result of step S5.8.

Specifically, semantics refers to more abstract features after operations of the convolution layer, the normalization layer, the activation layer and the pooling layer. After passing through the dropout layer and the fully connected layer, the fused semantic feature passes through Softmax to output the probability of each node which falls between [0,1] and is denoted as p₁∈R^{d}. p₁ indicates the occurrence probability of each type of reaction templates predicted according to the fused semantic feature. Furthermore, the ECFP4 semantic feature and the SMILES word one-hot semantic feature pass through the dropout layer, the fully connected layer and Softmax, respectively, so as to output the probabilities of each node which fall between [0, 1] and are denoted as p₂∈R^{d} and p₃∈R^{d}, respectively. p₂ and p₃ indicate the occurrence probabilities of each type of reaction templates predicted according to the ECFP4 semantic feature and SMILES word one-hot semantic feature respectively.

p₁ is a final output result of the network, which refers to the occurrence probability of each type of templates predicted according to the fused semantic features. The fused semantic feature is obtained by concatenating the ECFP4 semantic feature and the SMILES word one-hot semantic feature. The occurrence probabilities of each type of templates obtained by learning the ECFP4 semantic feature and the word one-hot semantic feature are p₂,p₃. In this way, in the training of the model, the ability of the network to learn the ECFP4 semantic feature and the SMILES word one-hot semantic feature is enhanced, so as to obtain the ECFP4 semantic feature and the SMILES word one-hot semantic feature which are more abstract. The fused semantic feature which is more abstract is obtained by concatenating the two features. That is to say, the network learns the fused semantic features and also learns the ECFP4 semantic feature and the SMILES word one-hot semantic feature. In this way, the ability of the ECFP4 feature and the SMILES word one-hot semantic feature expressing molecules can be enhanced, and the ability of the fused semantic features expressing molecules can also be enhanced, thus improving precision of the prediction result of the network.

Referring to FIG. 2, a diagram of a SMILES word one-hot feature according to the present disclosure is shown. Product Molecule denotes a product molecule, One-Hot-Encoding denotes one-hot-encoding, SMILES String denotes a SMILES string of a target product molecule, Word Vector denotes a word vector, and Composition of Word Vector denotes a composition of word vector, One-hot encoding of the SMILES string denotes a feature after one-hot encoding of the SMILES string, and SMILES Word One-Hot Feature denotes a word one-hot feature.

In the specific embodiment, in step S5, the multi-semantic network includes one input layer, six convolution layers, six normalization layers, six activation layers, six pooling layers, two dropout layers, three fully connected layers and three output layers.

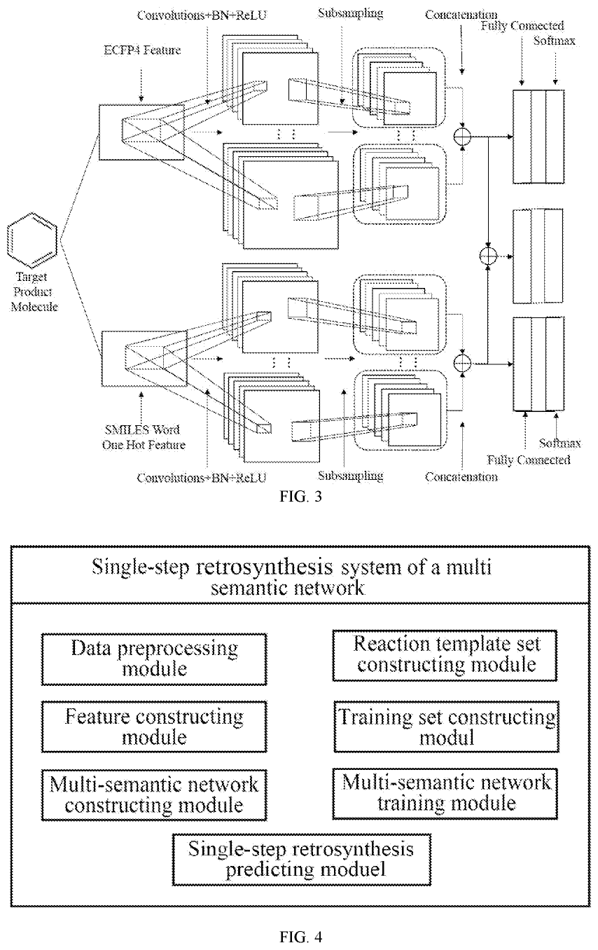

Referring to FIG. 3, a schematic diagram of a multi-semantic network according to the present disclosure is shown. Target ProductMolecule represents the target product molecule, and ECFP4 Feature and SMILES Word One Hot Feature represent the ECFP4 feature and the SMILES word one-hot feature of the target product molecule, respectively. Convolution+BN+ReLU represents convolution, normalization and activation operations, Subsampling represents subsampling, Concatenation represents concatenation, and Fully connected represents full connected.

In step S5.2, the ECFP4 feature input at the node 1 is convolved by using 3 convolution kernels with a size of 1×4096, in which a number of output channels of 3 convolution kernels is 100, so as to obtain the convolved ECFP4 feature. In step S5.3, the SMILES word one-hot matrix input at the node 2 is convolved by using 3 convolution kernels with a size of 3×120, 4×120 and 5×120, in which a number of output channels of 3 convolution kernels is 100, so as to obtain the convolved SMILES word one-hot matrix feature.

The ReLU activation function in step S5.5 is:

f(x)=max(0,x)

where x represents input of neurons, which can change all negative values to 0, while keeping the positive values unchanged. The unilateral inhibition function enables neurons in the neural network to have sparse activation.

In step S5.8, Softmax function is specifically defined as:

$${S}_{i} = \frac{{e}^{i}}{{Σ}_{j} {e}^{j}}$$

where e is a natural constant, Σⱼe^{j} represents a sum of powers of all neurons with e as a base and with the neuron as the index, and Sᵢ represents a result of a i-th neuron passing through Softmax.

In an embodiment, in the training process of step S6, according to three classification results of the model, three cross entropy losses of the obtained model are denoted as loss₁, loss₂ and loss₃, respectively, and a final loss of the single-step retrosynthesis prediction model is:

loss=α₁loss₁+α₂loss₂+α₃loss₃

where loss₁, loss₂ and loss₃ represent a predicted loss of the fused semantic feature, a predicted loss of the ECFP4 semantic feature and a predicted loss of the SMILES word one-hot semantic feature, respectively, and αⱼ (j=1,2,3) represents a weight of the three losses loss₁, loss₂ and loss₃ in the global loss of the network, respectively, where Σαⱼ=1 and αⱼ∈(0,1).

Specifically, the loss function represents the difference between a prediction result and a real value. The embodiment proposes a new loss function, which evaluates the classification result (classification probability P₁) obtained by fusion semantic learning originally, and additionally evaluates the classification results (classification probabilities P₂ and P₃) of the ECFP4 semantic feature and the SMILES word one-hot semantic feature. By comprehensively evaluating the whole model with weights α₁, α₂ and α₃, the training precision of the model is improved. The value of αⱼ is a decimal between (0,1), and the endpoints 0 and 1 are not taken.

In the specific implementation process, an Adam optimizer is used in the model. When the model is trained, three cross entropy losses are calculated according to the three output results of step S5.

The specific form of the cross entropy loss function lossⱼ (j=1,2,3) is as follows:

$${Loss}_{j} = - \frac{1}{d} {\sum }_{i} {\sum }_{c = 1}^{d} {y}_{i , c} log ( {p}_{j , i , c} )$$

where d is a total number of labels, that is, a size of the reaction template set T; y_{i,c} is a binary identifier, which indicates whether a real label of sample i is c, that is, whether a predicted rule of the sample i is the same as a real rule c, and 1 is taken when the real label of the sample i is the same as c, otherwise 0 is taken; p_{j,i,c} represents a j-th output probability of the network of the sample i with the label as c, that is, the j-th output probability of the sample i predicted by the network with the rule of c.

In the specific embodiment, in step S6, a number of model training epochs is set as 100, and multiple iterations are performed in each epoch until all training samples participate in training for one time, and a number of training samples batch size participating in one iteration is set as 128. The initial learning rate is set as 0.001.

The following specific examples illustrate and verify the method of the present disclosure.

Example 1: a publicly available chemical reaction data set USPTO-50k is preprocessed according to step S1; a reaction template set is constructed according to step S2; a ECFP4 feature and a SMILES word one-hot feature are constructed according to step S3; a set G is constructed according to step S4, and the set G is randomly divided into a training set, a verification set and a test set according to a ratio of 8:1:1. The training set and the verification set are used to train and select models, and the test set is used to perform prediction by the single-step retrosynthesis prediction model after training. The training set and the verification set are used to train the model, and the test set tests the prediction precision of the trained single-step chemical retrosynthesis prediction model. Table 1 shows prediction performance of the single-step retrosynthesis prediction method based on the multi-semantic network of the present disclosure in single-step chemical retrosynthesis. At present, the prediction precision of top-1, top-3, top-5 and top-10 of the best experimental results in the field is 52.5%, 69.0%, 75.6% and 83.7%. Apparently, the prediction precision based on the model of the present disclosure is significantly higher than the best results in the field at present.

TABLE 1
Prediction performance of single-step chemical
retrosynthesis of the multi-semantic network

| Top-1 | Top-3 | Top-5 | Top-10 |
|---|---|---|---|
| 61.8% | 80.6% | 85.1% | 89.5% |

Example 2: a publicly available metabolic reaction data set MetaNetX is preprocessed according to step S1; a reaction template set is constructed according to step S2; a ECFP4 feature and a SMILES word one-hot feature are constructed according to step S3; a set G is constructed according to step S4, and the set G is randomly divided into a training set, a verification set and a test set according to a ratio of 8:1:1. The training set and the verification set are used to train and select models, and the test set is used to perform prediction by the single-step retrosynthesis prediction model after training. The training set and the verification set train the model, and the test set tests the prediction precision of the trained single-step chemical retrosynthesis prediction model. Table 2 shows the prediction performance of the single-step retrosynthesis prediction method based on the multi-semantic network according to the present disclosure in single-step bioretrosynthesis. There are few existing researches on single-step bioretrosynthesis, and the model of the present disclosure can predict single-step bioretrosynthesis without matching enzyme information.

TABLE 2
Prediction performance of single-step bioretrosynthesis
of the multi-semantic network

| Top-1 | Top-3 | Top-5 | Top-10 |
|---|---|---|---|
| 47.0% | 66.4% | 73.4% | 79.8% |

Compared with the prior art, the embodiments of the present disclosure has the following beneficial effects.

1. The single-step retrosynthesis prediction method is a first method for performing single-step retrosynthesis prediction by using a multi-semantic fusion network in the field of single-step retrosynthesis, and is a template-based single-step retrosynthesis method. The prediction result has relatively high interpretability.

2. The present disclosure designs a new loss function, which can improve the training precision of the model by comprehensively evaluating three prediction results of learning of the multi-semantic network.

3. The present disclosure designs a semantic extraction method, which can extract deep semantic information of the ECFP4 feature and the SMILES word one-hot feature of target product molecules.

4. The present disclosure designs a construction method of a SMILES word one-hot feature, which can contain more potential information.

5. The single-step retrosynthesis prediction model of the present disclosure can be used as both single-step chemical retrosynthesis prediction and single-step bioretrosynthesis prediction, and matching operation of enzyme information is not required when the single-step bioretrosynthesis prediction is carried out.

### Embodiment 2

Based on the same inventive idea, the embodiment provides a single-step retrosynthesis system based on a multi-semantic network, which includes a data set preprocessing module, a reaction template set constructing module, a feature constructing module, a sample set constructing module, a multi-semantic network constructing module, a multi-semantic network training module and a single-step retrosynthesis predicting module.

The data set preprocessing module is configured to acquire a public data set and preprocess the public data set to obtain a preprocessed data set D, each piece of data in the data set D corresponds to one specific reaction, and each piece of data includes a reaction, a reactant molecule and a product molecule.

The reaction template set constructing module is configured to construct a reaction template set T.

The feature constructing module is configured to obtain an ECFP4 feature set E of a product molecule represented by an ECFP4 vector and a SMILES word one-hot feature set S of a product molecule represented by a SMILES word one-hot matrix, respectively, according to the target product molecule in the data set D.

The sample set constructing module is configured to construct a sample set G={(eᵢ, sᵢ), tᵢ}ᵢ₌₁^{N}, where eᵢ∈E and sᵢ∈S represent the ECFP4 feature and the SMILES word one-hot feature of the product molecules in a i-th data of the data set D, respectively, tᵢ∈T represents a reaction template in the i-th data of the data set D, and N represents a number of sample sets.

The multi-semantic network constructing module is configured to construct a multi-semantic network, the multi-semantic network includes an input layer, a convolution layer, a normalization layer, an activation layer, a pooling layer, a dropout layer, a fully connected layer and an output layer. The convolution layer is configured to convolve the input data, the normalization layer is configured to normalize the convolved feature, the activation layer is configured to activate the normalized feature, and the pooling layer performs pooling operation on the ECFP4 feature and the SMILES word one-hot feature, respectively, so as to obtain the ECFP4 semantic feature and the SMILES word one-hot semantic feature; fuse the ECFP4 semantic feature with the SMILES word one-hot semantic feature to obtain the fused semantic features; and then, pass the fused semantic features through the dropout layer, the fully connected layer and Softmax, to obtain the final output result by the output layer.

The multi-semantic network training module is configured to train the multi-semantic network in the multi-semantic network constructing module by using the sample set of the sample set constructing module to obtain a trained single-step retrosynthesis prediction model.

The single-step retrosynthesis predicting module is configured to, for a target product molecule to be predicted, use the single-step retrosynthesis prediction model trained in the multi-semantic network training module to predict a reaction template capable of generating the target product molecule, and finally calculate the SMILES string of the reactant molecule corresponding to the target product molecule in combination with the SMILES string of the target product molecule, thereby realizing single-step retrosynthesis prediction.

Referring to FIG. 4, a schematic diagram of a module of a single-step retrosynthesis system of a multi-semantic network according to an embodiment of the present disclosure is shown.

Generally speaking, the data set preprocessing module is configured to preprocess the data set to obtain the processed data set. The reaction template set constructing module is configured to generate a reaction template set based on the preprocessed data set. The feature constructing module is configured to generate an ECFP4 feature and a SMILES word one-hot feature of a product molecule according to the product molecule. The sample set constructing module is configured to generate a sample set consisted of the ECFP4 feature, the SMILES word one-hot feature and the reaction template. The multi-semantic network constructing module is configured to construct a multi-semantic network for single-step retrosynthesis prediction. The multi-semantic network training module is configured to train the multi-semantic network by using data in the sample set to obtain a single-step retrosynthesis prediction model of the trained multi-semantic network. The single-step retrosynthesis predicting module is configured to perform single-step retrosynthesis prediction on new target product molecules by using the multi-semantic network model.

Because the system introduced in Embodiment 2 of the present disclosure is the system used to implement the single-step retrosynthesis method based on a multi-semantic network in Embodiment 1 of the present disclosure, those skilled in the art can understand the specific structure of the system based on the method introduced in Embodiment 1 of the present disclosure, which will not be described in detail herein. All systems used in the method of Embodiment 1 of the present disclosure belong to scope to be protected by the present disclosure.

It should be understood that the above description of the preferred embodiments is more detailed, which should not be considered as a limitation on protection scope of the present disclosure. Under inspiration of the present disclosure, those skilled in the art can also make substitutions or modifications without departing from the protection scope claimed by claims of the present disclosure, all of which fall within the protection scope of the present disclosure. The claimed protection scope of the present disclosure shall be subject to the appended claims.

What is claimed is:

1. A single-step retrosynthesis method based on a multi-semantic network, comprising:
S1: acquiring a public data set, and preprocessing the public data set to obtain a preprocessed data set D, wherein each piece of data in the data set D corresponds to one specific reaction, and each piece of data comprises a reaction, a reactant molecule and a product molecule;
S2: extracting reaction templates from all data in the data set D by using an RDChiral tool, and removing repeated reaction templates, to obtain a final reaction template set T, wherein each reaction template contains one or more reactions;
S3: obtaining an ECFP4 feature set E of product molecules represented by ECFP4 vectors and a SMILES word one-hot feature set S of the product molecules represented by a SMILES word one-hot matrix, respectively, according to the product molecules in the data set D;
S4: constructing sample setsG={(eᵢ, sᵢ), tᵢ}ᵢ₌₁^{N}, where eᵢ∈E and sᵢ∈S represent the ECFP4 feature and the SMILES word one-hot feature of a product molecule in a i-th data of the data set D, respectively, tᵢ∈T represents a reaction template in the i-th data of the data set D, and N represents a number of the sample set;
S5: constructing the multi-semantic network, wherein the multi-semantic network comprises an input layer, a convolution layer, a normalization layer, an activation layer, a pooling layer, a dropout layer, a fully connected layer and an output layer, the convolution layer is configured to convolve input data, the normalization layer is configured to normalize a convolved feature, the activation layer is configured to activate a normalized feature, and the pooling layer performs pooling operation on the ECFP4 feature and the SMILES word one-hot feature, respectively, so as to obtain an ECFP4 semantic feature and a SMILES word one-hot semantic feature; fusing the ECFP4 semantic feature with the SMILES word one-hot semantic feature to obtain a fused semantic feature; and passing the fused semantic feature through the dropout layer, the fully connected layer and Softmax, to obtain a final output result by the output layer;
S6: training the multi-semantic network in S5 by using the sample sets in S4 to obtain a trained single-step retrosynthesis prediction model;
S7: for a target product molecule to be predicted, predicting a reaction template capable of generating the target product molecule by using the trained single-step retrosynthesis prediction model in S6, and calculating a SMILES string of the reactant molecule corresponding to the target product molecule by using the RDChiral tool in combination with the SMILES string of the target product molecule, thereby realizing single-step retrosynthesis prediction.

2. The method according to claim 1, wherein the S3 comprises:
according to the product molecules in the data set D, generating the ECFP4 vectors of the product molecules in all data in the data set D by using the RDKit tool to obtain the ECFP4 feature set E of product molecules represented by ECFP4 vectors; generating the SMILES word one-hot matrix of the product molecules in all data in the data set D by using a Sklearn tool to obtain the SMILES word one-hot feature set S of the product molecules represented by the SMILES word one-hot matrix.

3. The method according to claim 2, wherein the generating the SMILES word one-hot matrix of the product molecules in all data in the data set D by using a Sklearn tool to obtain the SMILES word one-hot feature set S of the product molecules represented by the SMILES word one-hot matrix comprises:
S3.1: performing one-hot-encoding on each character of an alphabet constructing the SMILES string to generate a word vector with dimension w₂; using word vectors of a first l₂ characters in each product molecule SMILES string to form a SMILES word one-hot matrix s₂∈{0,1}^{l}^{₂}^{×w}^{₂}, wherein if the product molecule SMILES string has less than l₂ characters, the product molecule SMILES string is padded with 0 vector;
S3.2: deeming every successive n rows in the matrix s₂∈{0,1}^{l}^{₂}^{×w}^{₂} as a group, the n rows corresponding to word vectors of n characters; concatenating the word vectors in a same group in sequence to obtain a composition of word vector with a length of w₁, w₁=n*w₂, a total of l₁ composition of word vectors being obtained,
$${l}_{1} = \frac{{l}_{2}}{n} ;$$
and constituting the SMILES word one-hot feature of the product molecules∈{0,1}^{l}^{₁}^{×w}^{₁}, where w₂, l₂ and n are positive integers, and n<l₂.

4. The method according to claim 1, wherein the multi-semantic network in S5 has one input layer, k₁+k₂ convolution layers, k₁+k₂ normalization layers, k₁+k₂ activation layers, k₁+k₂ pooling layers, two dropout layers, three fully connected layers and three output layers, where k₁ and k₂ are positive integers,
processing step in S5 comprises:
S5.1: inputting the ECFP4 feature represented by the ECFP4 vector at input node 1, and inputting the SMILES word one-hot feature represented by the SMILES word one-hot matrix at input node 2, wherein the input layer comprising the input node 1 and the input node 2;
S5.2: convolving the ECFP4 feature input at the input node 1 by using k₁ convolution kernels with a same size to obtain a convolved ECFP4 feature, wherein a number of output channels of the k₁ convolution kernels is c₁;
S5.3: convolving the SMILES word one-hot feature input at the input node 2 by using k₂ convolution kernels with different sizes to obtain a convolved SMILES word one-hot feature, wherein a number of output channels of the k₂ convolution kernels is c₂;
S5.4: normalizing the convolved feature in S5.2 and the convolved features in S5.3 by the normalization layer, respectively, to obtain a normalized ECFP4 feature and a normalized SMILES word one-hot feature;
S5.5: performing ReLU activation operation on the normalized ECFP4 feature and the normalized SMILES word one-hot feature by the activation layer, respectively, to obtain an activated ECFP4 feature and an activated SMILES word one-hot feature;
S5.6: performing max-pooling operation on the activated ECFP4 feature and the activated SMILES word one-hot feature by the pooling layer, respectively, to obtain a pooled ECFP4 feature and a pooled SMILES word one-hot feature;
S5.7: concatenating the pooled ECFP4 feature to obtain a concatenated ECFP4 semantic feature, concatenating the pooled SMILES word one-hot feature to obtain a concatenated SMILES word one-hot semantic feature, and concatenating the ECFP4 semantic feature and the SMILES word one-hot semantic feature to obtain fused semantic features;
S5.8: feeding the fused semantic features to one fully connected layer, and passing the fused semantic features through Softmax, to output probability of each node which falls between [0,1] and is denoted as p₁∈R^{d}, p₁ indicating a predicted occurrence probability of each type of reaction templates; passing the ECFP4 semantic feature and the SMILES word one-hot semantic feature through the dropout layer, the fully connected layer and Softmax, respectively, to output probabilities of each node which fall between [0, 1] are denoted as p₂∈R^{d} and p₃∈R^{d}, respectively, p₂ and p₃ indicating occurrence probabilities of each type of reaction templates predicted according to the ECFP4 semantic feature and SMILES word one-hot semantic feature, d being a number of the reaction templates in a reaction template set T;
S5.9: obtaining a final predicted result by the output layer according to a result in S5.8.

5. The method according to claim 1, wherein in the training process of step S6, according to three classification results of the model, three cross entropy losses of the model are denoted as loss₁, loss₂ and loss₃, respectively, and a final loss of the single-step retrosynthesis prediction model is:
loss=α₁loss₁+α₂loss₂+α₃loss₃
where loss₁, loss₂ and loss₃ represent a predicted loss of the fused semantic feature, a predicted loss of the ECFP4 semantic feature and a predicted loss of the SMILES word one-hot semantic feature, respectively, and αⱼ (j=1,2,3) represents weights of the three losses loss₁, loss₂ and loss₃ in a global loss of the network, respectively, where Σαⱼ=1 and αⱼ∈(0,1).

6. A single-step retrosynthesis system based on a multi-semantic network, comprising:
a data set preprocessing module configured to acquire a public data set and preprocess the public data set to obtain a preprocessed data set D, wherein each piece of data in the data set D corresponds to one specific reaction, and each piece of data comprises a reaction, a reactant molecule and a product molecule;
a reaction template set constructing module configured to construct a reaction template set T;
a feature constructing module is configured to obtain an ECFP4 feature set E of product molecules represented by ECFP4 vectors and a SMILES word one-hot feature set S of product molecules represented by a SMILES word one-hot matrix, respectively, according to the product molecules in the data set D;
a sample set constructing module configured to construct sample setsG={(eᵢ, sᵢ), tᵢ}ᵢ₌₁^{N}, where eᵢ∈E and sᵢ∈S represent the ECFP4 feature and the SMILES word one-hot feature of a product molecule in a i-th data of the data set D, respectively, tᵢ∈T represents a reaction template in the i-th data of the data set D, and N represents a number of sample set;
a multi-semantic network constructing module configured to construct the multi-semantic network, wherein the multi-semantic network comprises an input layer, a convolution layer, a normalization layer, an activation layer, a pooling layer, a dropout layer, a fully connected layer and an output layer, the convolution layer is configured to convolve input data, the normalization layer is configured to normalize a convolved feature, the activation layer is configured to activate a normalized feature, and the pooling layer performs pooling operation on the ECFP4 feature and the SMILES word one-hot feature, respectively, so as to obtain the ECFP4 semantic feature and the SMILES word one-hot semantic feature; fuse the ECFP4 semantic feature with the SMILES word one-hot semantic feature to obtain a fused semantic feature; and pass the fused semantic feature through the dropout layer, the fully connected layer and Softmax, to obtain a final output result by the output layer;
a multi-semantic network training module configured to train the multi-semantic network in the multi-semantic network constructing module by using the sample set of the sample set constructing module to obtain a trained single-step retrosynthesis prediction model;
a single-step retrosynthesis predicting module configured to, for a target product molecule to be predicted, predict a reaction template capable of generating the target product molecule by using the trained single-step retrosynthesis prediction model in the multi-semantic network training module, and calculate a SMILES string of the reactant corresponding to the target product molecule in combination with the SMILES string of the target product molecule, thereby realizing single-step retrosynthesis prediction.
